(19) United States
(12) Patent Application Publication
(10) Pub. No.: US 2026/0294335 A1
(43) Pub. Date: Oct. 1, 2026

(54) DEVICE AND METHOD FOR DETECTING SLEEP APNEA IN A PATIENT

(71) Applicant: Université du Luxembourg, ESCH-SUR-ALZETTE (LU)

(72) Inventors: Maria Gabriela RETAMALES BARAONA, Heisdorf (LU); Jorge Manuel MENDES SILVA GONCALVES, Belvaux (LU); Marino GAVIDIA, Dipach (LU)

(73) Assignee: Université du Luxembourg, ESCH-SUR-ALZETTE (LU)

(21) Appl. No.: 19/094,479

(22) Filed: Mar. 28, 2025

(51) Int. Cl. A61B 5/00; A61B 5/113; A61B 5/1455

(52) U.S. Cl. CPC A61B 5/4818; A61B 5/113; A61B 5/1135; A61B 5/14551; A61B 5/4809; A61B 5/7264

(57) ABSTRACT

A device for detecting sleep apnea comprising an abdominal movement sensor, an oxygen saturation sensor, a data acquisition unit configured to acquire an abdominal movement time-series from the abdominal movement sensor and an oxygen saturation time-series from the oxygen saturation sensor, and a sleep apnea detection unit configured to determine sleeping interval, Apnea-Hypopnea events, and an Apnea-Hypopnea Index.

### FIELD OF DISCLOSURE

The present invention relates to a device for detecting sleep apnea in a patient and to a method for detecting sleep apnea in a patient. More particularly, the present invention relates to a device and a method designed such that sleep apnea can be detected with a reduced number of sensors.

### BACKGROUND

Obstructive sleep apnea (OSA) is a sleep-related breathing disorder. The severity of OSA is diagnosed by the Apnea-Hypopnea Index (AHI), which indicates the number of times a person has apnea or certain types of hypopnea per hour of sleep, as defined by the American Academy of Sleep Medicine (AASM). An apnea is defined as a nasal flow reduction of more than 90%, while an hypopnea is defined as a nasal flow reduction of more than 30%, both for at least 10 s. There are several types of hypopneas, in which only the following two count towards AHI: hypopneas with a reduction of oxygen desaturation equal to 3% or more within 45 s and hypopneas with an arousal within 5 s after the end of the event. All other hypopneas are not considered in the calculation of AHI. According to the AASM guidelines, patients are categorized according to their AHI: healthy (less than 5 events/h), mild (5-15 events/h), moderate (15-30 events/h), and severe (more than 30 events/h).

OSA is diagnosed today by a polysomnography (PSG). PSG requires the over-night recording and monitoring of several physiological signals, including electroencephalo-gram (EEG), electro-cardiogram (ECG), electrooculogram, chin muscle activity, leg movements, respiratory effort, nasal airflow, and oxygen saturation (SpO2). This information is then used to compute the AHI.

Currently, there is only one published method that uses machine learning (ML) to estimate AHI using an oximetry signal (Levy, J., Álvarez, D., Del Campo, F. & Behar, J. Deep learning for obstructive sleep apnea diagnosis based on single channel oximetry. Nat. Commun. 14, 4881 (2023)).

When focusing only on short time-series segments, there are many classification tools to decide whether an apnea event is present or not. Such detection tools for OSA classification have been based on manual feature extraction, from time and/or frequency domains of one or more physiological signals (Ramachandran, A. & Karuppiah, A. A survey on recent advances in machine learning based sleep apnea detection systems. Healthcare 9, 914 (2021); Mendonca, F., Mostafa, S. S., Ravelo-Garcia, A. G., Morgado-Dias, F. & Penzel, T. A review of obstructive sleep apnea detection approaches. IEEE J. Biomed. Health Inform. 23, 825-837 (2018); Mostafa, S. S., Mendonça, F., G. Ravelo-García, A. & Morgado-Dias, F. A systematic review of detecting sleep apnea using deep learning. Sensors 19, 4934 (2019)).

The object of the present invention is providing a device and a method for detecting sleep apnea, which comprise a simplified handling and reduce the costs for the diagnosis.

### SUMMARY

In one aspect, the present invention pertains to a device for detecting sleep apnea in a patient, the device comprising an abdominal movement sensor for the patient, an oxygen saturation sensor for the patient, a data acquisition unit and a sleep apnea detection unit, wherein a first signal connection connects the abdominal movement sensor to the data acquisition unit and a second signal connection connects the oxygen saturation sensor to the data acquisition unit, the data acquisition unit being configured to acquire an abdominal movement time-series from abdominal movement sensor and an oxygen saturation time-series from the oxygen saturation sensor and to provide the abdominal movement time-series and the oxygen saturation time-series to the sleep apnea detection unit, the sleep apnea detection unit being configured to determine an AHI for the patient based on the abdominal movement time-series, the oxygen saturation time-series and a time-series from at most one additional sensor of different type.

In another aspect, the present invention pertains to a method for detecting sleep apnea in a patient, the method comprising at least the following steps: attaching an abdominal movement sensor, an oxygen saturation sensor and at most one additional sensor of different type to the patient; acquiring an abdominal movement time-series with the abdominal movement sensor, an oxygen saturation time-series with the oxygen saturation sensor and the time-series from the at most one additional sensor of different type with a data acquisition unit while the patient sleeps; and determining an AHI for the patient based on abdominal movement time-series, the oxygen saturation time-series and the time-series from the at most one additional sensor of different type with a sleep apnea detection unit.

### BRIEF DESCRIPTION OF THE DRAWINGS

FIG. 1a shows a schematic drawing of a patient with a first embodiment of the device.

FIG. 1b shows a schematic drawing of a patient with a second embodiment of the device.

FIG. 1c shows a schematic drawing of a patient with a third embodiment of the device.

FIG. 1d shows a schematic drawing of a patient with a fourth embodiment of the device.

FIG. 2 shows a schematic drawing of the components of the device.

FIG. 3 shows a schematic flow diagram of the method.

### DETAILED DESCRIPTION

The present invention provides a device for detecting sleep apnea in a patient with a reduced number of sensors and sensor data. Due to the reduced number of sensors and sensor data, the detection of sleep apnea is simplified and the costs for the device can be reduced.

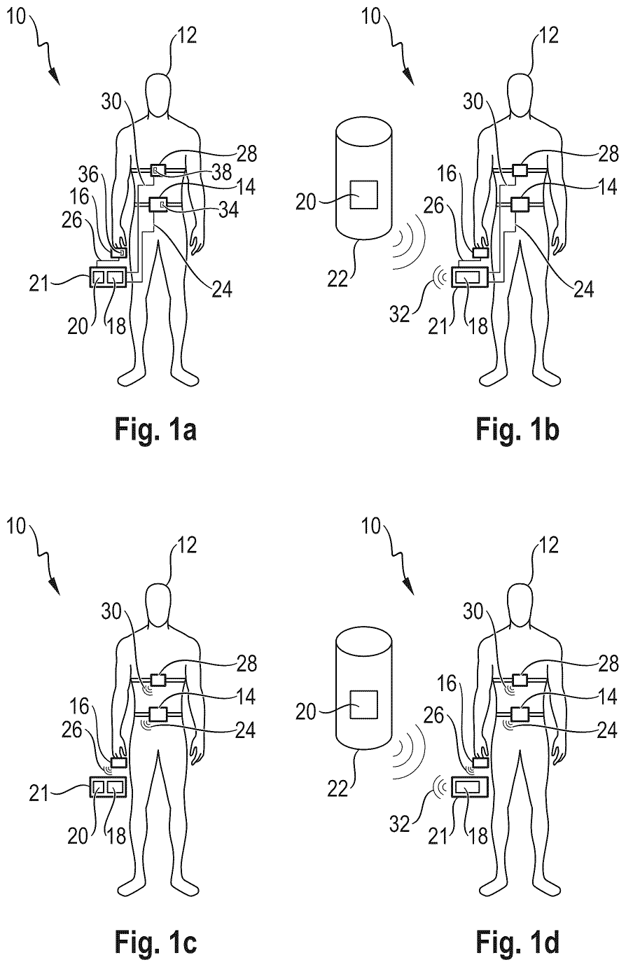

According to FIG. 1a, which shows a first embodiment of device 10, device 10 only requires two sensors at patient 12, an abdominal movement sensor 14 and an oxygen saturation sensor 16 to detect sleep apnea in patient 12. Device 10 is configured to provide an AHI based on data from the abdominal movement sensor 14 and the oxygen saturation sensor 16. To improve the accuracy of the AHI, the device 10 may use data from at most one additional sensor 28 having a sensor type different from the abdominal movement sensor 14 and the oxygen saturation sensor 16, for example a thoracic movement sensor. Hence, the at most one additional sensor 28 of different type is optional.

The abdominal movement sensor 14 may for example comprise a belt which the patient 12 can wear when asleep. The abdominal movement sensor 14 may provide an abdominal movement time-series 34 as measurement data from the patient 12.

Furthermore, the oxygen saturation sensor 16 may be attached to the patient's finger when the patient 12 is asleep. The oxygen saturation sensor 16 may provide an oxygen saturation time-series 36 as measurement data from the patient 12. The oxygen saturation sensor 16 may for example be a pulse oximetry sensor.

The thoracic movement sensor may also comprise a belt which the patient 12 can wear when asleep. Furthermore, the thoracic movement sensor may provide a thoracic movement time-series as the time-series 38 from the at most one additional sensor of different type. The time-series 38 is the measurement data from the patient 12. Furthermore, the device 10 can also determine an AHI without using the at most one additional sensor 28 or the time-series 38.

Furthermore, the device 10 may comprise a data acquisition unit 18 and a sleep apnea detection unit 20. A first signal connection 24 can connect the abdominal movement sensor 14 to the data acquisition unit 18. A second signal connection 26 can connect the oxygen saturation sensor 16 to the data acquisition unit 18. Furthermore, a third signal connection 30 can connect the utmost one additional sensor 28 of different type to the data acquisition unit 18.

In the first embodiment shown in figure la, the first, second and third signal connections 24, 26, 30 comprise signal cables extending from the abdominal movement sensor 14, the oxygen saturation sensor 16, and, if present, the at most one additional sensor 28 to the data acquisition unit 18, respectively.

The data acquisition unit 18 is configured to provide the acquired timed-series 34, 36, 38 from the abdominal movement sensor 14, the oxygen sensor 16 and the at most one additional sensor 28 to the sleep apnea detection unit 20. The sleep apnea detection unit 20 is configured to determine an AHI for the patient 12 based on the abdominal movement time-series and the oxygen saturation time-series. Furthermore, the sleep apnea detection unit 20 may use additional data to determine the AHI based on the time-series from the at most one additional sensor 28 of different type to improve the accuracy of the detection.

In the first embodiment, the sleep apnea detection unit 20 and the data acquisition unit 18 may be combined in one device, for example a mobile device 21. The mobile device 21 may for example be a smartphone.

In a second embodiment according to FIG. 1b, the mobile device 21 may only comprise the data acquisition unit 18. A fourth signal connection, being a wireless signal connection, may connect the mobile device 21 to an external server 22. The external server 22 may comprise the sleep apnea detection unit 20. Furthermore, the external server 22 may be located at a position being different to that of the mobile device 21. For example, the external server 22 may be located in another room, in another building, in another country and/or in another jurisdiction than the mobile device 21, i.e. the patient 12. The mobile device 21 may provide the time-series 34-38 from the abdominal movement sensor 14, the oxygen sensor 16 and the at most one additional sensor 28 to the external server 22 via the fourth signal connection 32.

The sleep apnea detection unit 20 may then determine the AHI. The external server 22 may then provide the determined AHI to the mobile device 21 via the fourth signal connection 32. The patient 12 can then assess the AHI and discuss the result with a physician.

In another embodiment, the external server 22 may provide the AHI to a database for the physician or to the physician directly.

In a third embodiment according to FIG. 1c and in a fourth embodiment according to FIG. 1d, at least one of the signal connections 24, 26, 30 connecting the abdominal movement sensor 14, the oxygen sensor 16, and the at most one additional server sensor 28 to the data acquisition unit 18 maybe wireless. The embodiment in FIG. 1c shows all signal connections 24, 26, 30 to be wireless. It is also conceivable, that only one of those signal connections 24, 26, 30 may be wireless and the remaining ones may be cable connections.

The explanations above concerning the first and second embodiment according to FIGS. 1a and 1b apply to the third and fourth embodiment according to FIGS. 1c and 1d in an analogous manner.

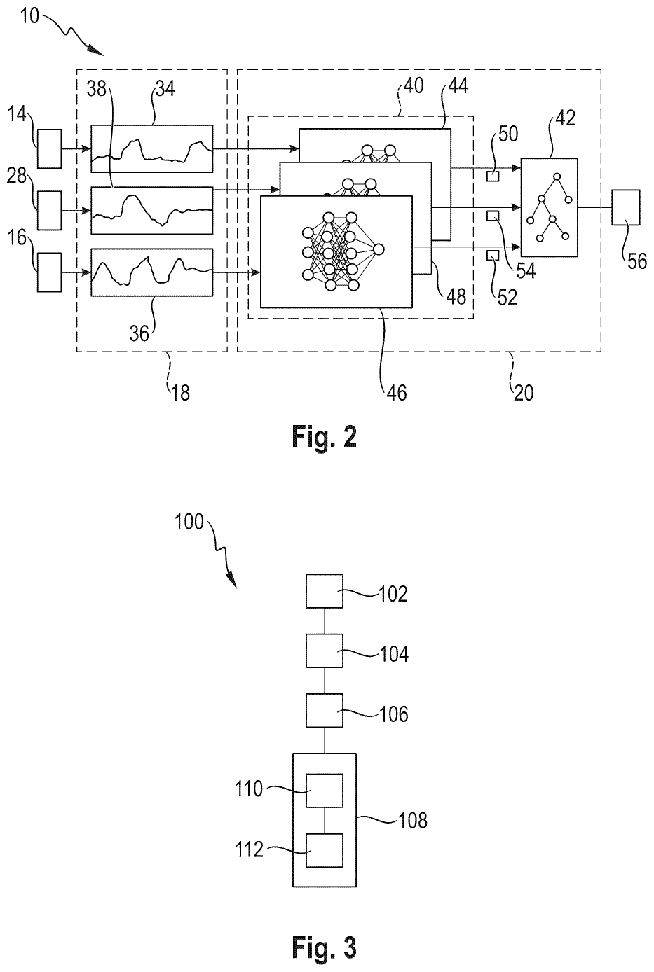

FIG. 2 shows the determination of the AHI 56.

The abdominal movement sensor 14 may provide the measured abdominal movement time-series 34 to the data acquisition unit 18. Furthermore, the oxygen saturation sensor 16 may provide the measured oxygen saturation time-series 36 to the data acquisition unit 18.

If at most one additional sensor 28 is provided, the at most one additional sensor 28 may provide an additional time-series 38 to the data acquisition unit 18.

The data acquisition unit 18 may provide the collected time-series 34-38 to the sleep apnea detection unit 20. The sleep apnea detection unit 20 may comprise a feature extractor 40 for analyzing the provided time-series 34-38.

The feature extractor 40 may comprise a first artificial neural network 44 having a first input level and a first output level. The first artificial neural network 44 may be trained such that it extracts first feature extraction data 50 from an abdominal movement time-series provided at the first input level and provides the first feature extraction data 50 at the first output level.

Furthermore, the feature extractor 40 may comprise a second artificial neural network 46 having a second input level and a second output level. The second artificial neural network 46 may be trained such that it extracts second feature extraction data 52 from an oxygen saturation time-series provided at the second input level and provides the second feature extraction data 52 at the second output level.

If the device 10 comprises at most one additional sensor 28 of different sensor type, the feature extractor 40 may comprise a third artificial neural network 48 having a third input level and a third output level. The third artificial neural network 48 may be trained such that it extracts the third feature extraction data 54 from a time-series from the at most one additional sensor 28 provided at the third input level. Furthermore, the third artificial neural network 48 may provide the third feature extraction data 54 at the third output level.

The abdominal movement time-series 34 is provided to the first artificial neural network 44 to determine first feature extraction data 50 of the abdominal movement time-series 34. The oxygen saturation time-series 36 is provided to the second artificial neural network 46 to determine second feature extraction data 52 of the oxygen saturation time-series 36. In case the at most one additional sensor 28 is present, the time-series 38 of the at most one additional sensor 28 is provided to the third artificial neural network 46 to determine third feature extraction data 54 of the time-series of the at most additional sensor 28.

The first, second and third feature extraction data 50-54 may comprise information about periods in which the patient is asleep and in which the patient is awake. Furthermore, the feature extraction data 50-54 may comprise information whether sleep apnea happened within periods in which the patient is asleep.

Furthermore, the sleep apnea detection unit 20 may divide the first, second and third feature extraction data 50-54 in periods of at most 80 seconds, preferably of at most 60 seconds, most preferably of at most 30 seconds. In that case, for each period, the feature extraction data 50-54 can comprise the information, whether the patient is asleep or awake and whether possible sleep apnea events happen in sleep periods. The periods may comprise an overlap with previous and/or following periods. The overlap may be at most 40 seconds, preferably at most 30 seconds, most preferably at most 15 seconds with at least one following or previous period.

The sleep apnea detection unit 20 may further comprise a classifier 42 which can assess the information provided by the first, second and third feature extraction data 50-54. The classifier 42 may be designed as a tree-based learning algorithm. The tree-based learning algorithm may be trained such, that it can determine, whether the combination of first feature extraction data 50 and the second feature extraction data 52 comprise Apnea-Hypopnea events. If the at most one additional sensor 28 is present, the classifier 42 may further be designed to consider the third feature extraction data 54 for the determination of Apnea-Hypopnea events.

The first feature extraction data 50, the second feature extraction data 52, and, if present, the third feature extraction data 54 can be provided to the classifier 42 in a concatenated form.

Based on the number of Apnea-Hypopnea events within the patient sleep periods, the sleep apnea detection unit 20 may determine the AHI 56. The classifier 42 may determine the AHI 56 via a regression algorithm based on the number of segments of the first, second and/or third feature extraction data 50-54. The segments of the feature extraction data 50-54 may for example be the periods of the feature extraction data 50-54 mentioned above. Alternatively, each segment may comprise at least two consecutive periods of the first, second and/or third feature extraction data 50-54 or a part of one period.

The classifier 42 may classify the segments as patient is asleep segments and patient is awake segments. Furthermore, the classifier 42 may classify any apnea event as nor-mal event or Apnea-Hypopnea event. Hence, both sleeping periods and sleep apnea events are determined with the time-series 34-38 of the sensors 16, 14, 28.

Based on that information, the sleep apnea detection unit 20 may determine the AHI.

The sleep apnea detection unit 20 can provide the AHI 56 to patient 12 for example via a display on the mobile device 21 and/or provide the AHI 56 to a physician or a database for physicians.

The evaluation of test patients has shown that using only the abdominal movement sensor and the oxygen saturation sensor, the sleep apnea detection unit can correctly classify 72.4% of all test patients into one of the four AHI classes: healthy (less than 5 events/h), mild (5-15 events/h), moderate (15-30 events/h), and severe (more than 30 events/h), with 99.3% either correctly classified or placed one class away from the true one. The device 10 provides a good trade-off between accuracy and patient's sleep comfort, when compared to a full polysomnography. Compared to a Polysomnographic Technologist's manual detection and diagnosis of OSA, computer-assisted signal analysis systems can reduce errors related to inter-and intra-operation variability and tiredness induced by the arduous an-notating process.

Moreover, the architecture of the sleep apnea detection unit, particularly the use of the first, second, and third artificial neural network 44-48 allows for each time-series 34-38 to have its own sampling frequency, and it estimates both AHI events and sleep stages. The best trade-off between model performance and patient comfort was achieved when combining data from only two sensors, abdominal movement sensor 14 and oxygen saturation sensor 16.

FIG. 3 shows a flow diagram of method 100 for determining the AHI 56. Method 100 can make use of the device 10 mentioned above.

According to first step 102, an abdominal movement sensor 14 and an oxygen saturation sensor 16 are attached to a patient 12. The abdominal movement sensor 14 can have a belt, which a patient 12 may wear around his body such that the abdominal movement sensor 14 is arranged above the abdomen. Furthermore, the oxygen saturation sensor 16 may comprise a finger clip which the patient 12 may clip on his finger. Furthermore, the oxygen saturation sensor 16 may comprise a finger ring which the patient 12 may put on his finger.

At most one additional sensor 28 of different type may be attached to the patient 12. The at most one additional sensor 28 may be a thoracic movement sensor, which may also have a belt. Thus, the patient 12 may wear the thoracic movement sensor on his body, wherein the thoracic movements sensor is arranged around the patient's chest. The use of the at most one additional sensor 28 is optional.

If the abdominal movement sensor 14, the oxygen saturation sensor 16 and/or the at most one additional sensor 28 require a cable line for sending time-series to a data acquisition unit 18, the sensors 14, 16, 28 may further be attached to the data acquisition unit 18 via cables. Those cables are the signal connections 24, 26, 30 between the corresponding sensors 14, 16, 28 and the data acquisition unit 18.

In another embodiment, a wireless signal connection can connect the abdominal movement sensor 14, the oxygen saturation sensor 16 and/or have at most one additional sensor 28 to the data acquisition unit 18.

While the patient 12 sleeps, the abdominal movement sensor 14, the oxygen saturation sensor 16, and the at most one additional sensor 28 may collect measurement time-series according to step 104. The abdominal movement sensor 14 may measure and collect abdominal movement time-series 34. The oxygen saturation sensor 16 may collect and measure oxygen saturation time-series 36. The at most one additional sensor 28, for example a thoracic movement sensor, may collect additional time-series, for example thoracic movement time-series.

The data acquisition unit 18 may for example be a mobile device 21. The mobile device 21 may further comprise a sleep apnea detection unit 21. In that case, the data acquisition unit 18 may provide the collected time-series 34, 36, 38 to the sleep apnea detection unit 20.

In another embodiment, the mobile device 21 may not comprise the sleep apnea detection unit 20. Instead, an external server 22 may comprise the sleep apnea detection unit 20. The external server 22 may be located on another location than the mobile device 21, for example in another room, in another building, in another country and/or in another jurisdiction.

The sleep detection unit 20 may estimate a patient sleep period according to step 106. For that estimation, the sleep detection unit 20 may analyze the abdominal movement time-series 34, the oxygen saturation time-series 36 and, if present, the time-series 38. The analysis may be performed by a combination of artificial neural networks 44-48 and a classifier 42 comprising a tree-based learning algorithm. The tree-based learning algorithm may be trained to decide whether the first feature extraction data 50 and the second feature extraction data 52 comprise Apnea-Hypopnea events. Furthermore, the tree-based learning algorithm may be trained to decide whether the third feature extraction data 58, if present, comprise Apnea-Hypopnea events.

The artificial neural networks 44 to 48 may be dedicated to extract features of time-series of one of the sensors 14, 16, 28. For example, the abdominal movement time-series 34 may be provided to the first artificial neural network 44. The time-series 36, 38 from the further sensors 16, 28 may have dedicated artificial neural networks 46, 48, in an analogous manner.

Furthermore, the time-series 34-38 may be divided in a plurality of periods, for example of at most 80 seconds, preferably at most 60 seconds, or most preferable at most 30 seconds. Hence, the sampling rate of the sensors 14, 16, 28 may be different, i. e. optimized to the requirements of the respective measurement. The periods may comprise an overlap with previous and/or following periods. The overlap may be at most 40 seconds, preferably at most 30 seconds, most preferably at most 15 seconds with at least one following or previous period.

According to sub step 110, the abdominal movement time-series 34, the oxygen saturation time-series 36 and, if present, the time-series 38 may be provided to input layers of the first artificial neural network 44, the second artificial neural network 46, and the third artificial neural network 48, respectively.

The feature extraction data 50-54 from the periods of the time-series 34-38 may be concatenated and then provided to the classifier 42. The tree-based learning algorithm of classifier 42 may then determine whether the periods are sleep periods and whether sleep apnea events are present in the feature extraction data 50-54. The determination may be performed based on the number of Apnea-Hypopnea events and the number of segments classified as sleep periods using a regression method.

Based on that information, the sleep apnea detection unit 20 may determine the AHI 56 according to sub step 112.

The example described above is by no means intended to limit the invention. Rather, the invention can be modified in many different ways. All the features of the invention described above may be essential to the invention, either alone or in combination with each other.

1. A device for detecting sleep apnea in a patient, the device comprising an abdominal movement sensor for the patient, an oxygen saturation sensor for the patient, a data acquisition unit and a sleep apnea detection unit, wherein a first signal connection connects the abdominal movement sensor to the data acquisition unit and a second signal connection connects the oxygen saturation sensor to the data acquisition unit, the data acquisition unit being configured to acquire an abdominal movement time-series from abdominal movement sensor and an oxygen saturation time-series from the oxygen saturation sensor and to provide the abdominal movement time-series and the oxygen saturation time-series to the sleep apnea detection unit, the sleep apnea detection unit being configured to determine an Apnea-Hypopnea Index for the patient based on the abdominal movement time-series, the oxygen saturation time-series and at most one time-series from at most one additional sensor of different type.

2. The device according to claim 1, wherein the device comprises the at most one additional sensor of different type.

3. The device according to claim 2, wherein the at most one additional sensor of different type is a thoracic movement sensor.

4. The device according to claim 1, wherein the sleep apnea detection unit is further configured to detect a patient sleep period based on the abdominal movement time-series, the oxygen saturation time-series and the at most one time-series from the at most one sensor of different type, or wherein the device further comprises a server, preferably at a location being different from the data acquisition unit, the server comprising the sleep apnea detection unit, or wherein the first signal connection and/or the second signal connection comprise a wireless signal connection.

5. The device according to claim 1, wherein the sleep apnea detection unit comprises a first artificial neural network being trained to provide first feature extraction data when provided the abdominal movement time-series and a second artificial neural network being trained to provide second feature extraction data when provided the oxygen saturation time-series.

6. The device according to claim 5, wherein the sleep apnea detection unit further comprises a classifier being configured to determine the Apnea-Hypopnea index from the first feature extraction data and the second feature extraction data.

7. The device according to claim 6, wherein the classifier comprises a decision tree configured to decide whether the first feature extraction data and the second feature extraction data comprise Apnea-Hypopnea events.

8. (canceled)

9. The device according to claim 1, wherein the device further comprises a mobile unit, the mobile unit comprising the data acquisition unit.

10. The device according to claim 9, wherein the mobile unit is a smartphone or wherein the mobile unit comprises the sleep apnea detection unit.

11-14. (canceled)

15. A method for detecting sleep apnea in a patient, the method comprising at least the following steps:
attaching an abdominal movement sensor, an oxygen saturation sensor and at most one additional sensor of different type to the patient;
acquiring an abdominal movement time-series with the abdominal movement sensor, an oxygen saturation time-series with the oxygen saturation sensor and a time-series from the at most one additional sensor of different type with a data acquisition unit while the patient sleeps; and
determining an Apnea-Hypopnea Index for the patient based on abdominal movement time-series, the oxygen saturation time-series and the time-series from the at most one additional sensor of different type with a sleep apnea detection unit.

16. The method according to claim 15, wherein the at most one additional sensor of different type is a thoracic movement sensor, and the method further comprises estimating a patient sleep period based on the abdominal movement time-series, the oxygen saturation time-series and the time-series from the at most one sensor of different type.

17. (canceled)

18. The method according to claim 15, wherein the abdominal movement time-series, the oxygen saturation time-series and the at most one time-series from the at most one sensor of different type each are divided into at least two different periods.

19. The method according to claim 18, wherein the sleep apnea detection unit estimates each period as being a patient sleep period or a patient awake period, wherein determining the Apnea-Hypopnea Index preferably is based on the patient sleep periods, or wherein each period comprises at most 80 seconds, preferably at most 60 seconds, most preferably at most 30 seconds, or wherein each period comprises an overlap of at most 40 seconds, preferably at most 30 seconds, most preferably at most 15 seconds, with at least one further period.

20-22. (canceled)

23. The method according to claim 15, wherein determining the Apnea-Hypopnea Index comprises the sub-step:
providing the abdominal movement time-series to an input layer of a first artificial neural network being trained to provide first feature extraction data when provided the abdominal movement time-series and providing the oxygen saturation time-series to a second artificial neural network being trained to provide second feature extraction data when provided the oxygen saturation time-series.

24. The method according to claim 23, wherein determining the Apnea-Hypopnea Index comprises the further sub-step:
determining at least one Apnea-Hypopnea event from the first feature extraction data and the second feature extraction data with a classifier.

25. The method according to claim 24, wherein the classifier comprises a decision tree configured to decide whether the first feature extraction data and the second feature extraction data comprise Apnea-Hypopnea events.

26. (canceled)

27. The method according to claim 15, wherein the abdominal movement sensor, the oxygen saturation sensor and the at most one additional sensor of different type provide the abdominal movement time-series, the oxygen saturation time-series and the at most one time-series from the at most one sensor of different type to a data acquisition unit on a mobile unit.

28. The method according to claim 27, wherein the mobile unit is a smartphone, or wherein the mobile unit comprises the sleep apnea detection unit.

29. (canceled)

30. The method according to claim 28, wherein the data acquisition unit provides the abdominal movement time-series, the oxygen saturation time-series and the at most one time-series from the at most one sensor of different type to a server comprising the sleep apnea detection unit, wherein the server preferably is at a location being different from the data acquisition unit.

31. (canceled)

32. The method according to claim 30, wherein the server provides the Apnea-Hypopnea Index to the patient.
